(19) United States
(12) Patent Application Publication
(10) Pub. No.: US 2022/0193225 A1
(43) Pub. Date: Jun. 23, 2022

(54) COMPOSITIONS AND METHODS FOR SARS-2 VACCINE WITH VIRUS REPLICATIVE PARTICLES AND RECOMBINANT GLYCOPROTEINS

(71) Applicant: Bruce Lyday, Orange, CA (US)

(72) Inventor: Bruce Lyday, ORANGE, CA (US)

(21) Appl. No.: 17/408,361

(22) Filed: Aug. 20, 2021

Related U.S. Application Data
(60) Provisional application No. 63/103,910, filed on Aug. 31, 2020.

(51) Int. Cl. A61K 39/215; A61K 48/00; A61K 39/39; A61P 37/04; A61P 31/14

(52) U.S. Cl. CPC A61K 39/215; A61K 48/0066; A61K 2039/53; A61P 37/04; A61P 31/14; A61K 39/39

(57) ABSTRACT

A novel and improved vaccine for prevention of disease caused by the Severe Acute Respiratory Syndrome-2 (SARS-2), /COVID-19 virus. Current mRNA and Adenovirus vaccine technologies for SARS-2 provide high levels of serum Immunoglobin G (IgG), antibodies against the original Wuhan strain, but there are now hundreds of mutant strains which can evade both vaccine and convalescent antibodies. These vaccines also do not provide strong mucosal IgA class antibodies which provide wider protection against mutant strains of Flu A and other respiratory viruses. The ability of these technologies to provide high levels of protection is in question, as serum neutralizing antibodies may decline to undetectable levels after six months. The appearance of mutant strains such as the Beta, Gamma, Delta, and Epsilon strains, containing altered amino acid sequences capable of evading vaccine-induced antibodies, calls for new vaccine technologies that can be quickly altered to meet this threat. The following describes a combination approach to prevention of infection by SARS-2/COVID-19. This combination consists of a priming injection of Recombinant Replicative Particles (VRP) derived from the Alphavirus Venezuelan Equine Encephalitis (VEE) strain 3000/3526, with insertion of a Delta/B.1.617.2 SARS-2/COVID-19 spike 1 glycoprotein (gp)-Receptor-Binding Domain (RBD) gene. The insertion of Internal Ribosome Entry Sites (IRES), elements between the 26S promoter and the SARS-2/COVID RBD gene allows for more efficient translation of the SARS-2/COVID gene products. The VEE^{3000/3256} VRP are produced from plasmids, so while they are infectious for one replicative cycle in vivo, progeny VRP are replication incompetent. The priming is followed by one or more intranasal administrations of a suspension of recombinant SARS-2/COVID-19 envelope spike 1 glycoproteins (gp), from selected mutant strains, combined with the pulmonary surfactant adjuvant, SF-10. The goal of the invention is to safely provide multiple immune layers of protection in both the upper and lower respiratory tracts, with induction of both mucosal IgA and serum IgG antibodies, as well as effector Cytotoxic T Lymphocyte (CTL), cells recognizing conserved regions of the SARS-2/COVID-19 virus genome. Secondary goals are to reduce the risk of antibody-dependent enhancement (ADE), of infection, a major concern with other SARS-2/COVID-19 vaccine designs, and to provide capacity to protect against mutant emergent strains of SARS-2/COVID-19 with annual intranasal boosters of new spike glycoproteins.

### TABLE OF SEQUENCES IN ASCII FORMAT SUBMITTED VIA EFS-WEB PURSUANT TO 37 CFR 1.821

| | | Date of | Size of | |
|---|---|---|---|---|
| Name of ASCII File | Organism Name | Creation | File In Bytes | |
| VEE Complete Sequence | Virus, Family Alphaviridae, | Aug. 18, 2021 | 12 | Kb |
| ASCII.txt | Venezuelan Equine Encephalitis | | | |
| | Virus | | | |
| B.1.617.2 Delta Spike Sequence | Virus, Family Coronaviridae, subfamily | Aug. 18, 2021 | 5 | kb |
| text file ASCII.txt | Betacoronaviridae, Severe Acute Respiratory | | | |
| | Syndrome- 2 Coronavirus | | | |
| | Strain B.1. 617.2 (Delta) | | | |
| B.1.351 Beta Spike 1 Sequence | Virus, Family Coronaviridae, subfamily | Aug. 18, 2021 | 5 | kb |
| Text File ASCII.txt | Betacoronaviridae, Severe Acute Respiratory | | | |
| | Syndrome- 2 Coronavirus | | | |
| | Strain B.1.351 (Beta) | | | |
| P.1 Gamma Spike 1 sequence | Virus, Family Coronaviridae, subfamily | Aug. 18, 2021 | 5 | kb |
| ASCII.txt | Betacoronaviridae, Severe Acute Respiratory | | | |
| | Syndrome- 2 Coronavirus | | | |
| | Strain P.1 (Gamma) | | | |

### ABBREVIATIONS

ACE2 Angiotensin-converting enzyme-2 ADE Antibody-Dependent Enhancement APC Antigen-Presenting Cell ARDS Acute Respiratory Distress Syndrome BCG Bacillus-Calmette-Guerin BCR B-cell receptor BHK Baby hamster kidney BSL Biologic Safety Level C Capsid SARS-2/COVID-19 Novel Coronavirus-19 CTL Cytotoxic T Lymphocyte DC Dendritic Cell E Envelope EMEM Eagles Minimal Essential Media EV71 Enterovirus 71 GOI Gene of Interest GP Glycoprotein HA Hemagglutinin HAI Hemagglutinin Inhibition IFA Influenza A IgA Immunoglobin A IgG Immunoglobin G IgM Immunoglobin M IL Interleukin IN Intranasal IRES Internal Ribosome Entry Site M Matrix M1 Macrophage type 1 M2 Macrophage type 1 MCP-1 Macrophage Chemokine Protein-1 MERS Middle East Respiratory Syndrome mRNA messenger RNA NAB Neutralizing antibody NK Natural Killer NP Nucleocapsid Protein NTD N-Terminal Domain PRR Pattern Recognition Receptor RBD Receptor Binding Domain SARS Severe Acute Respiratory Syndrome S1 Spike 1 S2 Spike 2 TCID Tissue Culture Infectious Dose TLR Toll Like Receptor UTR Untranslated Region VEE Venezuelan Equine Encephalitis Virus VLP Virus-Like Particle Variant of Concern VOC VRP Viral Replicative Particle

### BACKGROUND OF THE INVENTION

This invention is in the field of preventative vaccines for viral diseases. Severe Acute Respiratory Syndrome Coronavirus (SARS-2/COVID-19), appeared on 12/12/19 in the city of Wuhan, China. SARS-2/COVID-19 represents a threat to public health and society of a magnitude similar to the Spanish Flu pandemic of 1918-1920 which killed an estimated 50 million people out of 500 million infections. There are several factors which contribute to the threat posed by SARS-2/COVID-19:
1. The appearance of mutant strains, with altered protein sequences capable of evading antibodies, more efficient transmission, or both.
2. Neutralizing Antibodies (NAB), to SARS-2/COVID-19s are short-lived, with a low % (16.7%), of patients retaining NAB at 60 days post-infection
3. The virus is stable in aerosol droplets and remains viable for 6-8 hours in air and up to 72 hours on plastic and stainless-steel surfaces
4. Unlike Influenza A, SARS-2/COVID-19 utilizes two spike proteins (S1 and S2), to attach to the host cell receptor ACE2. When S1 attaches, it induces a conformational change in the protein configuration, exposing S2, previously hidden from neutralizing antibodies, completing the binding and fusion process.
5. The binding strength of Spike to ACE2 is approximately 10-20-fold higher than that of SARS-1, possibly impacting the high rate of human-human community spreading.
6. Serum IgG Antibodies to SARS-2/COVID-19 Spike antigens may exacerbate acute lung injury and mortality in victims through ADE-immunopathology, and resulting inflammatory cytokine storm similar to secondary infections with dengue virus. This is documented with SARS-1, while not been proven for SARS-2/COVID-19, caution is warranted as the two viruses share many similarities.
7. There has been a documented rise in the incidence of heart inflammation (myo- and endo-carditis), after the 2″ shot of the mRNA vaccines. This may prove a problem with plans to continue booster injections of mRNA to protect against mutant strains.
8. The vaccines currently in use are designed to protect against the first Wuhan strain. While effective at preventing infection by the Wuhan and Alpha strains, other mutants have been shown to cause breakthrough infections in those fully-vaccinated against the Wuhan strain.
9. Serum IgG levels with current mRNA vaccines are generally high weeks after injection, but decline thereafter. Reports are circulating of titers of neutralizing antibodies declining to undetectable levels after six months. While memory B cells can still mount a recall response, this decline in protection is causing concern among public health and vaccine professionals.
10. There are now hundreds of mutant variant strains, including the Alpha (UK), Beta (South Africa), Delta (India), and Kappa (Brazilian). These all contain mutations within the spike 1 gp which either:
   a) Allows for tighter binding to the ACE2 receptor, increasing transmissibility and viral load
   b) Allows for evasion of convalescent, therapeutic, and vaccine-induced antibodies. These are located primarily in the RBD region, but have also been identified in the N-Terminal Domain (NTD)

SARS-2 Phylogeny

SARS-2/COVID-19 belongs to family Coronaviridae, subfamily Betacoronaviridae. SARS-2/COVID-19 is a large, enveloped, positive-strand RNA virus of approx. 120 nm in diameter. Its 31.6 kb genome is large for RNA viruses, and codes for the major structural protein products Nucleoprotein (NP), Matrix (M), Spike (S), and Envelope Protein (E). There are also several non-structural genes coding for enzymes including RNA-dependent RNA polymerase. Unique among RNA viruses, Coronaviruses carry a gene for a proofreading enzyme which limits mutations. Many types of Coronaviruses are implicated in cases of the common cold. The structural locations and purpose of the described proteins are:
NP—forms core protein complex with genomic RNA
M—defines shape of viral envelope and central organizer of viral assembly
S—Stalk protruding from membrane, it binds to the host cell receptor and serves as the major target for neutralizing antibody and is subdivided into an expressed (S1), and hidden (S2) protein.
E—a small glycoprotein important in viral envelope development.

Phylogenetically, SARS-2/COVID-19 is related to several other coronaviruses, some of which cause human disease. SARS-1 appeared in China in 2004, and caused severe upper and lower respiratory syndrome, with a mortality of 10-12%. SARS-2/COVID-19 shares 79.5% sequence homology with SARS-1, and the discovery of the ten-fold stronger binding of SARS-2/COVID-19 to the main receptor Angiotensin-converting enzyme 2 (ACE2), compared with SARS-1, may explain its rapid transmissibility. Other coronaviruses similar to SARS-2/COVID-19 include Middle Eastern Respiratory Syndrome (MERS) virus, which apparently crossed over from camels to humans in 2012, and had a mortality of 34.2%. SARS-2/COVID-19 shares 96.2% sequence homology with the bat virus CovRaTG13, and the discovery of SARS-2/COVID-19 RNA at the Wuhan market where wild-caught animals, including bats, are sold for food, provides a plausible link for the virus spread.

There are no specific treatments approved for SARS-2/COVID-19, although various drugs approved for other viral and non-viral diseases are being tested. The efficacy record for RNA antivirals, unless targeting unique enzymes such as those expressed by HIV, is low compared to antibiotics. Undoubtedly, vigorous exploration in these areas will continue, but success is by no means assured.

The most efficacious means of reducing or eliminating the spread of viruses is through the use of prophylactic (preventative) vaccine. Vaccines have not been successful in many cases, including HIV, but have virtually eliminated such diseases as smallpox and polio. A successful vaccine provokes protective immune responses to viral antigens (proteins of immunological significance). While no vaccine has proven 100% safe and effective, the vaccines in use today prevent millions of illnesses and countless deaths from infectious diseases each year. These responses of the adaptive immune system include specific antibodies which recognize, and bind to short amino acid (peptide) sequences on the exposed portions of the virus. They are proteins consisting of both heavy and light amino acid chains. One end is termed F^{ab} or antigen-binding, the other terminus is called F^{c}, which binds complement or the F^{c} receptor of macrophages or other effector cells. Antibodies are termed either neutralizing or non-neutralizing. Neutralizing antibody (NAB), can block virion-host cell receptor binding or fix complement for virion destruction. Antibodies are produced by mature B lymphocytes (plasma cells), after picking up antigen presented by Dendritic Cells through their B-cell receptor (BCR).

Emergence of Mutant Variants of Concern (VOC)

The SARS-2 Spike 1 contains a Receptor-Binding Domain (RBD), which binds to human ACE2 receptors. Mutant V.O.C. such as the Delta strain have been identified that combine antibody evasion with higher transmission capacity. These mutants are capable of causing breakthrough infections and illness even inf fully-vaccinated persons.

Antibody Types:
1. IgA or Immunoglobulin A, mucosal antibodies secreted by cells lining the mucus membranes. These antibodies are of special significance in prevention of respiratory infections, binding to the glycoproteins of virus spikes. Secretory IgA appears in a dimeric form, and is the dominant type. IgA are poor fixers of complement compared with serum IgG. In terms of production, secretory IgA is produced more than any other antibody type. Polymeric S-IgA appears to play a crucial role in protection against homologous and variant flu strains. A single intranasal vaccine of the SARS spike protein conferred protection in a mouse model.
2. IgM—These poly-antibodies are formed as pentamers of light and heavy chains, and appear early in response to infection (3-7 days).
3. IgG—Known as gamma-globulins, these specific, shorter antibodies appear 10-14 days after infection, and persist for long periods of time, though levels (titers), may decrease. The titer of serum IgG₃ is the one of the accepted correlates of protection from Influenza A.

The “gold standard” of protection against challenge with Influenza A is to achieve a titer of specific IgG3 to the HA antigen of the virion of at least 1:40 for 50% protection. While IgG3 antibodies to SARS-2/COVID-19 will definitely play a role in vaccines efficacy, there are significant challenges to a successful vaccine using IgG3 against the Spike (S1 and S2), protein antigens.

The first obstacle is the evidence that specific IgG3 recognizing SARS-2/COVID-19 declines rapidly. A study found that of 59 patients who recovered from SARS-2/COVID-19, only 16.7% had high (>ID₅₀ of 2000), levels of NAB after 60 days. A recent study of 243 health care workers in Japan given two shots of a mRNA vaccine found that levels of neutralizing antibody dropped to undetectable levels after six months. A vaccine which relies solely on NaB to spike proteins of SARS-2/COVID-19, like Influenza A, may fail to provide adequate protection.

Significant Factors in SARS-2/COVID-19 Vaccine Design

The second obstacle facing a vaccine design with a foundation based on NAB to SARS-2/COVID-19 Spike antigens (S-IgG), is safety. Antibodies, especially those in blood (serum IgG), can be protective or cause harm. Antibodies carry two receptors, the F^{ab} end attaches to the antigen, and the F^{c} end fixes complement for destruction of the virus, or engulfment by a macrophage. Many patients who die from SARS-2/COVID-19 infection present with Acute Lung Injury (ALI). Immunopathology appears to play in important role, with elevated levels of IL-6 and IL-8 creating a cytokine storm, damaging delicate alveoli and impairing oxygen uptake.

In 1967, researchers were surprised to find that 80% of children vaccinated with Respiratory Syncytial Virus (RSV), were hospitalized compared with 5% of unvaccinated children. A review of human patients with SARS-2/COVID-19 revealed that recovering patients developed NAB after 20 days, but patients who died developed them sooner at 14.7 days. This points to an aggravating effect of antibodies in ARDS cases. Evidence points to a damaging effect of serum IgG antibodies against S1/S2.

A Chinese macaque model of SARS-1 showed evidence that the presence of S-IgG from a vaccine increased the likelihood of ALI and lack of protection. Macrophages can exist as either M1 activated or M2 regulatory. The S-Ig attached to the virus attracted M1 macrophages through the Fc receptor. This is known as antibody-dependent enhancement (ADE), and is well-characterized in the viral infection caused by dengue.

This ADE induced high levels of the inflammatory MCP-1 chemokine and cytokines, including TNFα, IL-6, and IL-8. This activation abrogated the macrophages M2 wound-healing properties, so damage accumulated in the pulmonary spaces without the capacity for repair. This could explain the 20-25% of patients who recover from SARS-2/COVID-19 showing evidence of “ground glass” opacities seen on CT images.

In summary, it appears that the major mechanism conferring protection against Influenza A, serum IgG, could have both a protective and a detrimental role in SARS-2/COVID-19. A vaccine designed only to raise serum IgG titers against S1/S2 would likely fail to confer full protection, or even aggravate disease. An alternative to using full-length S1/S2 in a vaccine design is to use only the receptor-binding domain (RBD), of S1. Studies have shown that NAB against the RBD are very effective and do not carry the risk of ADE. For the intranasal component, there is value to using the full-length S1/S2 construct as mucosal IgA is not implicated in ADE. Clearly, a novel vaccine design is required to deal with this unusual phenomenon.

Another significant obstacle to a SARS-2/COVID-19 vaccine is the presence of >60 polysaccharide chains attached to the S1 complex. These can hinder antibody binding, and the high degree of glycosylation (although lower than SARS-2/COVID), contributes to the failure of vaccines against HIV.

An ideal vaccine design for SARS-2/COVID-19 would include:
Ability to induce high levels of both serum IgG and mucosal polymeric IgA which neutralize the virus without inducing ADE
Ability to induce high levels of CTL, especially CD4+ cells of effector-memory subset recognizing conserved epitopes of the SARS-2/COVID-19 genome
Provide protection against antigenic drift/shift mutant strains of SARS-2/COVID-19
Ability to induce protective immune responses in both the Upper and Lower Respiratory Tracts
Have a safety profile allowing administration to high-risk persons: children, the elderly
Be able to be tested using current transgenic murine and primate models
Have commercial scale-up capability

Current SARS-2/COVID Vaccine Technologies

Currently, there are at least six vaccines for SARS-2/COVID-19 in clinical trials, and many more in pre-clinical development. A live, attenuated version of SARS-2/COVID-19 would most likely have a high-risk potential for causing disease, and killed vaccines are often sub-optimal inducers of protective CTL responses. Given these factors, the six designs represent emergent technologies in the field of vaccine design. These can be classified as:
1) Vector designs. Vectors use either a virus (Adenovirus, Lentivirus, etc.), or another pathogen (BCG bacillus), to carry transgenes coding for the vaccine-target viral antigens. When inserted into the vector using recombinant DNA techniques, the new particle can be injected into the subject where it makes entry to the cytoplasm. After uncoating and translation, the desired antigens are expressed on replicative particles, or as secreted proteins. Immune responses to these new antigens include IgG, IgA, and CD4+ and CD8+ CTL, in addition to activation of innate effectors: Natural Killer (NK) cells and macrophages.
2) mRNA designs. mRNA vaccines consist of the messenger RNA segments coding for the viral antigens. These segments, after entering the host cell, are translated into viral proteins and released to generate a host immune response
3) Sub-unit vaccines. These consist of the antigenic/immunogenic proteins of the viral pathogen. While envelope glycoprotein spikes are often used, other virus gene products: nucleocapsid, matrix, etc., can also be used. These proteins, produced by E. coli or a Eukaryotic cell system for proper glycosylation, are often combined with an adjuvant to increase antigen uptake and presentation by Dendritic Cells (DC). Sub-unit vaccines are the only type of emergent vaccines to be granted approval (Hepatitis B).

Each of these systems have strengths and weaknesses. Proper vaccine design involves an intensive review and analysis of the known properties of the pathogen and the correlates of immunity (Neutralizing antibody titers, CTL type and numbers, etc.). After this exercise, a review of the available new vaccine and adjuvant technologies can be performed, and promising candidates for testing identified.

Vector Design Strengths:
These are most similar to live attenuated vaccines, as they trigger many Toll-Like Receptor (TLR), and Pattern Recognition Receptors (PRR) which activate the innate immune system. The helper-dependent vector Viral Replicative Particles (VRP) have the added safety factor or limiting recombination events to produce hybrid replicates capable of causing disease.
There are few limits to the type of gene product able to be cloned into a vector expression system, only the size of the segment constrains insertion.
VRP based on alphaviruses can selectively infect DC, leading to innate and adaptive B and T cell responses in both serum and the mucosal compartments.
Vectors have proven safe and effective in many animal studies, including SARS-1.

Vector Design Drawbacks:
Vectors can generate an inflammatory immune response in the subject. This can reduce immunity be suppressing transgene expression and have harmful and even fatal outcomes in the case of Adenovirus vectors.
Adenovirus vectors, while efficient expressors of transgenes, often have to overcome pre-existing levels of neutralizing antibodies, approaching 40% for USA subjects in the case of Ad5.
BCG based vaccines, while proven effective at generation of a “trained immunity” to other pathogens mediated by innate effectors in infants, have not been proven to generate protective immunity to heterologous viral pathogens in adults. These also lack the glycosylation (attachment of polysaccharide chains) to envelope spike proteins. This could alter the antibody response to the transgene product.
The immune response will be directed at both the vector and transgene products, diminishing the desired protective effect. A vector design using AdHu5 expressing HIV nucleocapsid and other transgenes failed to provide protection in a human trial.

mRNA Vaccine Strengths:
Flexibility and ease of scale. Clinical batches can be produced in weeks after obtaining the correct sequences.
Low cost and stability of final product compared with live, attenuated, especially important in remote areas with poor access to cold storage
The process is cell-free, eliminating many cumbersome steps (Benzonase, affinity chromatography, etc.) to filter unwanted debris from the final product with cell-based systems. Purification by HPLC is usually sufficient.
mRNA segments are easily packaged in Liquid Nanoparticles (LNP), which help in packaging and have adjuvant properties.
The self-amplifying properties of the mRNA constructs allow for efficient translation of desired antigens
The immune response is not weakened by the presence of other antigens as with vector systems

mRNA Vaccine Drawbacks:
Careful attention must be made to the specifics of the 5′7′ methylguanosine (m7G), cap construction. Failure can affect protein translation and immunogenicity
mRNA constructs can generate unwanted inflammatory responses
mRNA is a transient molecule by nature that is easily degraded by nuclease activity.
As the optimum LNP:mRNA mass ration may be 10:1 to 30:1, this limits multi-antigen constructs as an overuse of adjuvant can cause safety and tolerability problems.
Human trial data has been disappointing with low levels of NAB generated compared with live attenuated vaccines. A mRNA vaccine which generated Hemagglutinin Inhibition (HAI) to Influenza A of 10,000 in primates generated only 70 in humans.

Sub-Unit Vaccine Strengths:
Sub-unit vaccines, like mRNA constructs, have tremendous flexibility in the type of antigens
Sub-unit vaccines are stable and cost-efficient compared with live attenuated vaccines.
Sub-unit vaccines do not require cold-chain systems like attenuated vaccines
Sub-unit vaccines have a good safety profile in human trials and aftermarket use
Sub-unit vaccines have been approved for many human pathogens, including HPV and Hep B

Sub-Unit Vaccine Drawbacks:
Low immunogenicity due to small size and low valences of antigens
Poor induction of CTL immunity compared with live attenuated or inactivated vaccines
Titers of NAB tend to diminish over time
Sub-unit vaccines must be paired with the correct adjuvant for protection from challenge

Cytotoxic T Lymphocytes in Vaccine Design

T lymphocytes originate from c-kit⁺ Sca1⁺ hematopoietic stem cells (HSC) in the bone marrow. The cells migrate to the thymus where they undergo further selection into CD4⁺ and CD8⁺ cells. CD4⁺ cells are termed helper-inducer T cells, though they also have regulatory and cytolytic functions. CD4⁺ T cells recognize longer peptides of 12-24 amino acids presented by Class II MHC complexes on dendritic cells.

These cells act as critical accessory cells between Dendritic Cells, which process and present antigen, and effector cells CD8⁺ cytotoxic T cells (CTL) and B cells producing antibodies. This occurs mainly through contact-mediated activation in lymph tissue, and the secretion of cytokines: IL-2, IL-4, IL-7, IL-10, IL-12, IL-17, IL-17, IL-21, etc. When a CD4⁺ T cell received both antigen and activation signals, it develops cytolytic capacity and secretes IL-2, IL-7, IL-12, IL-15, and IL-21. When a CD4⁺ cell receives an antigenic signal in the absence of activation, it develops regulatory capacity, down-grading immune responses through secretion of IL-4 and IL-10. In this manner immune responses are tightly regulated.

CD8⁺ T cells (CTL), are sometime suppressor/regulatory, but most often assume a cytolytic function, recognizing short, 8-12 amino acid chains presented by Class I MHC present on most somatic cells. When a viral peptide is presented, the cytolytic machinery composed of perforin and Granzyme B destroys the target cell by perforation of the membrane and induction of apoptosis.

Together, CD4⁺ and CD8⁺ cells can provide protective immunity from viruses, either in the context of recovery from disease or through vaccination. The best-know model for CTL importance in vaccination is Influenza A. Studies show the importance of CD4⁺ cells as they recruit early innate system effectors into the lung to blunt virus infection, and promote strong, effective protection mediated by B and CD8⁺ T cells. These cells, which predominantly recognize peptides from the conserved NP and M proteins, reside in the memory pool after vaccination or infection. The recall response is noted for its strength and speed compared with primary challenge.

Models appear to prove the importance of CD4⁺ And CD8⁺ cells. A single immunodominant peptide (S525) from the SARS spike protein, protected 80% of C57/BL6 from SARS viral challenge. Humans with higher precursor frequency of CTL recognizing internal Flu virus proteins have greater protection than those making responses against the HA spike protein, which is not heavily conserved and subject to high mutation rates. Although internal proteins can mutate, these mutations are often fatal as they impair assembly of the core and adjacent proteins.

Types of Vaccines Currently Licensed for Use

The original vaccine was Vaccinia or cowpox virus. Dr. William Jenner made the astute observation that milkmaids were often immune to smallpox. Inoculation of non-pathogenic cowpox virus into humans raised Ab and CTL responses that were cross-protective against the more lethal smallpox.

The Types of Vaccines Currently in Use are Summarized Below:
1. Live, attenuated vaccines are composed of viruses which have been weakened by UV light, chemical mutagens, serial passage through semi-permissive cell lines, or other means. Examples include chickenpox, measles, mumps, and rubella. Live attenuated vaccines promote the strongest immune responses, as they replicate in the person and provoke both CTL and NAB responses. The drawbacks of live vaccines include their unsuitability for children, the elderly, and those with compromised immune systems.
2. Killed vaccines contain all of the virion proteins, but do not replicate in vivo. Examples include Hepatitis A, polio (Salk), and rabies. Killed vaccines are safer, but do not provoke strong CTL responses, and NAB responses are weaker compared with the live vaccines.
3. Sub-unit vaccines contain only protein antigens, usually with an adjuvant to increase their immunogenicity. Although safe, these vaccines are generally poor inducers of cellular immunity. An example is the Hepatitis B vaccine.

A live, attenuated vaccine against SARS-2/COVID-19 appears unlikely to be approved for testing due to safety concerns. A killed vaccine may have a higher margin of safety, but the lack of CTL protection is an obstacle, and the rapidly declining IgG titers to the virus make this approach problematic. Sub-unit vaccines may offer a degree of protection with a high margin of safety, but will require combination with an approach designed to induce strong CTL responses to have a chance at >50% protection of subjects. DNA and mRNA designs hold promise, but may provoke auto-immune responses. Vector designs are safer than live or killed vaccines, but pre-existing antibodies to the vector and the inefficiency of GOI translation can reduce the desired immune effect.

In summary, it appears that current vaccine technologies in approved use have serious deficiencies when applied to SARS-2/COVID-19. A novel and improved approach is needed, and is described herein.

### BRIEF SUMMARY OF INVENTION

The invention is comprised of two components, a viral replicative vector carrying a SARS-2 transgene administered by hypodermic syringe, and a mixture of SARS-2 variant spike proteins in a surfactant adjuvant administered by intranasal inhalation. The invention is designed to provide a safe method of inducing powerful blood antibody (IgG), mucosal antibody (IgA), and T cell responses against mutant strains of SARS-2. Currently, mRNA and vector vaccine designs can provide IgG protection which is diminished after several months, limited T cell responses, and poor IgA responses.

### DETAILED DESCRIPTION OF INVENTION

Vaccine Component #1

VEE VRP Expressing SARS-2/COVID-19 Spike 1 RBD (Prime)

Construction of VRP Consisting of VEE^{3000/3526} with SARS-2/COVID-19 RBD Gene Insertions.

A new form of vaccine offering the protection of a live attenuated vaccine and the safety of a killed vaccine is the recombinant viral vector design. Recombinant viral vectors use genetic engineering to insert foreign transgenes into the vector genome. The transgenes are then produced by the host cell as viral proteins capable of inducing an immune response. Alphaviruses are small, enveloped RNA viruses of family Togaviridae, subfamily Alphaviridae. Examples include Sindbis, Venezuelan Equine Encephalitis (VEE), and Semliki Forest Virus. Of these, attenuated strains of VEE transformed into recombinant vectors have been tested in human volunteers with an acceptable safety record in cancer immunotherapy trials.

VEE has some unique attributes for use as a vaccine vector. First, existing Neutralizing antibody (NaB), to VEE is very rare outside the NE region of South America. Second, VEE has a cell tropism for Dendritic Cells (DC), which act as central regulators of the immune system. DC of the CD11b infected with VEE-VRP migrate to lymph nodes to prime powerful CTL and antibody responses through interactions with CD4⁺ helper/inducer subsets and CD4⁺ follicular helper cells which sustain strong, long-lasting antibody responses to viral pathogens.

Some more advantages of VEE-VRP are that the use of a bipartite helper-plasmid construction allows for in vitro assembly of infectious VEE particles. These particles, when injected into humans, are capable of infecting DC, but the progeny particles are antigenic/infectious but replication-incompetent. This induces a powerful yet safer immune response than a replication-competent vector. Another advantage is the use of Internal Ribosome Entry Sites (IRES) from a virus such as the human Enterovirus EV71. This allows for more efficient translation of the foreign gene, increasing the antigenicity and resulting immune response.

There are Three Main Types of Vector Systems in Use:
1. Naked RNA uses in vitro transcribed RNA from an expression vector virus consisting of the viral NS replicase genes and the foreign gene inserted downstream of the strong sub-genomic 26s promoter. These systems lack viral structural genes are incapable of replication.
2. Recombinant particles (VLP/VRP), use in vitro transcribed RNA from an expression vector virus and a helper vector supplying viral structural proteins into mammalian cells like BHK-21. This system has the advantage of glycosylation (added polysaccharide chains) to envelope glycoproteins. The generated particle is good for one round of replication, then stops. This promotes a strong immune response similar to that of live, replicating virus, without the danger of multiple infection cycles. VRP preferentially infect Dendritic Cells and macrophages, with consequent cytokine activation and efficient generation of specific CTL and B cells producing IgG. VRP engineered to infect DC are efficient as generation of mucosal IgA, critical for efficient protection against respiratory viruses such as Influenza A and SARS.
3. Layered DNA vectors—The layered DNA vector system consists of delivery of a DNA vector providing foreign gene expression from a CMV or similar promoter, replacing the SP6 RNA polymerase promoter of the Alphavirus.

The Venezuelan Equine Encephalitis (VEE^{3000/3526}) Virus Recombinant Particle (VRP)

Venezuelan Equine Encephalitis (VEE) is a medium size (70 nm), enveloped RNA virus of family Togaviridae, subfamily Alpphaviridae. The virus is transmitted by the bite of an infected Aedes mosquito, and causes potentially fatal encephalitis in horses and humans. Bovine, Canine and Porcine species can be infected, but do not show symptoms. There are six different serologically distinct subtypes and numerous strains of VEE. The largest recent outbreak occurred in Colombia in 1995, with over 14,000 cases and 26 reported deaths.

VEE has been the subject of research for both vaccine and biowarfare projects. The TC-83 strain, developed by USAMRID at Ft. Detrick, Md., is available to military and other persons serving in high-risk areas. The TC-83 strain is manufactured by 83 serial passages in guinea pig cardiac cells.

Several members of Alphaviridae, including VEE, are preferred platforms for recombinant vector systems to express foreign viral antigens in a VRP particle. These can have the advantages of high immunogenicity and safety as they are replication-restricted. The vectors can be constructed using the parent sequence of VEE³⁰⁰⁰ to produce the VEE³⁵²⁶ VRP platform. The advantages of the VEE³⁵²⁶ platform is that while the original VEE³⁰⁰⁰ strain is highly immunogenic, it can only by assembled in Biosafety Level-3 (BSL-3), facilities. The VEE³⁵²⁶ strain is prepared by deletion of the furin cleavage site in the Envelope 3 (E3) gene [Δ56RKRR59], and a 2^{nd} site resuscitation in E1.

The recombinant VRP offer significant advantages to Naked RNA and Layered systems for viral vaccines, as they mimic pathogenic virus strains to a certain degree (glycosylation of envelope glycoproteins when expressed in mammalian cell lines, one round of replication). Among VRP, the VEE^{3000/3526} platform represents a wild-type strain with robust vaccine capabilities due to its superior DC-infecting capabilities, among other advantages. These include the generation of recombinant proteins, production of VLP, and in vivo efficacy as a vaccine against emergent viruses. It also can be constructed in BSL-2 conditions, increasing scale-up capability for production.

Construction of Split Helper VEE^{3000/3526} VRP Vectors

In a split-helper vector design, a second copy of the 265 promoter is inserted into the genome either immediately upstream of the authentic promoter or between the E1 gene and the beginning of the 3′ untranslated region. A foreign gene of interest (GOD, is then inserted into the genome just downstream of the second 26S promoter such that a second sub-genomic mRNA containing the foreign gene is transcribed. For added translation of the GOI, an IRES sequence cloned from Enterovirus 71 (EV71), can be inserted between the 265 promoter and the GOI.

The EV71 IRES element (strain 7423/M5/87) can be PCR amplified from pdc/MS DNA using primers dc/M5 (EcoRI) F and dc/MS (BamHI) R. The EV71 IRES PCR product is then digested with EcoRI and BamHI restriction enzymes and ligated into the VEE³⁰⁰⁰ VRP-RBD and plasmids downstream of the 265 promoters and upstream of the SARS-2/COVID gene sequences.

These VEE vectors replicate in infected cells under GMP conditions and assemble into infectious particles. These particles, when injected into humans, can infect DC, but progeny particles are replication incompetent as they lack the two helper plasmids for complete VRP construction. When such vectors are based on vaccine strains of alphaviruses, they can be utilized in vivo for immunization against both the alpha-virus vector and the pathogen from which the heterologous gene was derived. The use of the VEE capsid and the VEE glycoprotein on two separate helper RNAs reduce the probability of recombination events by a factor of 10 e4.

To construct a VEE^{3000/3526} vector that can be manufactured in BSL-2 conditions, deletion of the entire furin cleavage site between VEE E3 and E2 can be performed, with a secondary site resuscitation mutation in E1 that allows production in a mammalian cell line such as Vero or BHK-21. These modifications prevent possible reversions-to-virulence in the mammalian cell. This new system uses sequences of the wild-type VEE strain, including the 5′ and 3′ untranslated regions (UTR).

The viral capsid and glycoprotein genes are inserted into separate helper plasmid constructs between the 26S subgenomic promoter and the start of the 3′ UTR. After linear alignment of the three plasmid constructs are tied by ligase, the RNA transcripts are electroporated or transfected into BHK-21 cells or another suitable cell line. Cell culture supernatants are then harvested by pipetting, then filtered by ultra-centrifugation through 60 nm Millipore filters. Filtered VRP particles are then measured for titer by plaque assay on Vero E6 cells using serial ten-fold dilutions and calculation of viral plaques after 48 hours and 72 hours.

The following contains the materials and methodology used to construct and test the VEE 3526 VRP clones (VEE^{3000/3526} VRP-SARS-2/COVID-RBD), containing the sequences of the SARS-2/COVID-19 RBD sequence.

Plasmid Construction and Insertion of SARS-2/COVID-19 Genes

An example of construction of the two recombinant VEE VRP particles, each carrying a structural gene from SARS-2/COVID-19, is described below.

In order to insert the desired gene (Spike 1-RBD for SARS-2/COVID-19, the complete genomes of VEE 3000 must be cloned. The parent VEE 3000 is derived from the Trinidad Donkey strain of VEE (GenBank L01442.2 Genebake VEE TDS). The VEE cDNA is downstream from a T7 RNA polymerase promoter so that linearization of the clone downstream of the VEE sequences, and subsequent in vitro transcription with T7 polymerase, yields infectious VEE genomic replicas. Plasmid SARS-2/COVID-19-RBD is constructed using a T7 promoter, containing the complete RBD sequence of the Delta strain of SARS-2/COVID-19 Spike-1 RBD (parent sequence Genbank 01K504), and is used to produce VEE³⁵²⁶-SARS-2/COVID-19-RBD. This sequence is located from nt #21481 to 25325 and is listed in the accompanying ASCII text file “B.1.617.2 Delta Spike Sequence Text File”.

The VEE replicon is prepared from a plasmid carrying a complete cDNA copy of the VEE genome modified to contain a second 26S promoter followed by a multiple cloning site from Cla12 adaptor plasmid. The insertion of EV71 IRES sequences downstream of the 26S promoter and upstream of the SARS-2/COVID transgene allows for more efficient translation. The double promoter clone is digested with ApaI, which cleaves within the 265 promoters bracketing the structural protein genes. Re-ligation reconstitutes a single 265 promoter followed by a multiple cloning site, which is used to insert the heterologous SARS-2/COVID-19 gene fragment. For insertion of these plasmids, a shuttle vector is used.

The helper constructs are derived from the pVEE³⁰⁰⁰ clone by partial deletion of the genes encoding the VEE nonstructural proteins. When necessary, incompatible 5′ and 3′ overhanging ends are made blunt by treatment with T4 DNA polymerase prior to re-ligation of the plasmid.

The bipartite helper system consisted of individual Capsid (C)- and glycoprotein (GP)-helper RNAs which are constructed from VEE^{3000/3526} 520±7505. In the C-helper, nt 8495±11229 are deleted by digestion of VE³⁰⁰⁰ A 520±7505 with HpaI and religation of the 3.8-kb DNA fragment. In the GP-helper, nt 7565±8386 are deleted by digestion of VEE³⁰⁰⁰ 520±7505 with Tth111I and SpeI followed by ligation of the 5.7-kb DNA fragment with the synthetic double-stranded oligonucleotide 5′-TAGTCTAGTCCGCCAAGATGTCA-3′. This oligonucleotide contained Tth111I and SpeI overhanging ends at the 5′ and 3′ ends, respectively, and reconstituted the 265 promoter downstream from the Tth111I site, the initiation codon normally used for the capsid protein, and the first codon of E3.

Transcription and Transfection

Plasmid templates are linearized by digestion with NotI at a unique site downstream from the VEE³⁰⁰⁰ cDNA sequence, and capped run-off transcripts were prepared in vitro with the RiboMAX T7 RNA polymerase kit. BHK cells are transfected by electroporation and incubated in 75-cm² flasks at 37° C. in 5% CO₂. For the preparation of VRP, transcripts of both the replicon and the helper plasmids were co-electroporated into BHK cells, and the culture supernatants were harvested at 30 hrs. after transfection.

Analysis by Western Blot of fractionated VRP harvested from transfected culture supernatants can be performed to confirm expression of the SARS-2/COVID-19 genes. Alternatively, monoclonal antibodies with GFP-tags can be utilized on whole VRP for the spike protein, and on sonically fractionated VRP for the nucleocapsid protein.

Scale-Up and Purification

For large-scale production of VRP, BHK or other suitable cell lines (Vero E6, e.g.), can be expanded by serial culture passage into Master and Working Cell Bank systems after appropriate tests confirm absence of pathogens. Cells from the Working Bank can then be expanded in successively larger flasks, then transferred to roller bottles with supplemented EMEM media. When 80-90% confluent, these roller bottles can be inoculated with the VRP for production.

Cells and supernatant are then removed and purified by standard means (Benzonase treatment, DNAase, Tangential Flow Filtration sucrose density gradient centrifugation), to remove unwanted cell debris. The final VRP particles can then be titered by plaque assay and have antigens confirmed by ELISA and Western Blot. The two VRP types are then combined in a 50/50 mixture for final fill and finish.

Storage and Administration

After titer has been determined by plaque assay, the VRP clones can be stored at −20° C. after lyophilization for reconstitution with EMEM and sterile water prior to administration. Alternatively, the VRP can be stored in a preservative (15% Trehalose sugar, 2% F127 surfactant, and 2% Human Serum Albumin, e.g.), and stored cold at 2-4° C.T. The titer of virus equal to the appropriate dose determined by animal studies then can be administered by hypodermic injection to the optimum site for maximum immune activation and safety. These dosages and site of injection will be determined by results of applicable animal models using young, healthy, and aged or immunosuppressed animals.

Vaccine Component #2 SARS-2/COVID-19 Spike1 Glycoprotein+SF10 Adjuvant Intranasal Administration (Boost)

Review

While the prime component of the novel vaccine design may be successful in generation of high titers of neutralizing IgG antibodies and CTL against SARS-2/COVID-19 virus, a strong mucosal immune response is critical to providing the first line of defense against respiratory viruses. In order to achieve this goal, the “boost” strategy seeks to elevate the immune response induced by the prime phase. To achieve multiple layers of protection in both the Upper and Lower respiratory tracts, intranasal administration of SARS-2/COVID-19 antigens follows the “prime” injection.

Intranasal administration has been used with success against the Influenza A virus. Several preclinical studies on adjuvant-combined, nasal-inactivated vaccines revealed that nasal S-IgA Abs, a major immune component in the upper respiratory tract, reacted with homologous virus hemagglutinin (HA) and were highly cross-reactive with viral HA variants, resulting in protection and cross-protection. Serum-derived IgG Abs, which are present mainly in the lower respiratory tract, are less cross-reactive and cross-protective.

Inactivated influenza vaccines induce both S-IgA and IgG Ab responses in the respiratory tract when administered intranasally with an appropriate adjuvant. In addition, one clinical study demonstrated that the ability of human S-IgA Abs to neutralize influenza viruses increased with increasing polymerization of IgA (IgA Abs can form dimers, trimers, tetramers, and larger polymers). This suggests that polymeric S-IgA plays a crucial role in protecting against both homologous and variant influenza viruses.

The literature suggests that mucosal IgA Abs are secreted actively, whereas mucosal IgG Abs diffuse from the serum to the mucus. These results also support previous findings that S-IgA Abs play a primary role in preventing influenza virus infection in the upper respiratory tract, whereas serum IgG Abs play a predominant role in preventing the progression to lethal influenza-induced pneumonia in the lower respiratory tract.

Thus, the ability of S-IgA Abs to provide cross-protection depends on polymeric structures, which displayed increasing neutralization activity with increasing polymerization. These results suggest that the presence of large polymeric S-IgA Abs with higher neutralization activity in the respiratory tract play a crucial role in providing protection against homologous and variant influenza viruses.

While the “prime” component of the described novel vaccine should be capable of inducing mucosal immune responses, a “boost” regimen may be required in order to increase the levels of large, polymeric S-IgA antibodies for more complete protection against SARS-2/COVID-19. The equivalent antigen to Influenza A HA is the SARS-2/COVID-19 spike glycoprotein.

The SARS-2/COVID-19 S gp is a trimeric protein with two sub-components, S1 and 52. These have multiple glycosylation sites branching from Asparagine residues. As the binding of amino acids to sugars is less favorable than other proteins, these sites represent a further obstacle to vaccine developers. S1 is expressed in the free virion, and upon binding of the Receptor-Binding Domain (RBD), a conformational change is triggered from a “down” to an “up” position. This allows S2 to be exposed, increasing the binding force to the cellular Angiotensin-converting enzyme-2 (ACE2) receptor. This has implications for vaccine developers, as antibodies directed against S2 may not be able to access their binding site when in the “down” position.

The use of various cells to produce viral gp for vaccine subunits has been employed, but mammalian cells are preferred as they can attach the saccharide chains required to duplicate the viral gp structure and antigenicity. The following described a method of production and purification of SARS-2/COVID-19 Spike 1/2 glycoproteins using a Vaccinia (Cowpox), VRB12 and pRB21 plasmid viral vector system expressed in a CV-1 (green monkey kidney) cell line.

Materials Required
1. vRB12 virus.
2. pRB21 plasmid.
3. CV-1 cells.
4. Serum-free Dulbecco's modified Eagle's medium (DMEM) (cellgro).
5. 2×DMEM (GIBCO).
6. 100× Penicillin/Streptomycin (cellgro).
7. 1.5% Noble Agar/DW (distilled water) (Difco).
8. 1% Neutral Red/DW (Sigma).
9. PCK Trypsin (Sigma).
10. DNase (Promega).
11. 1% Crystal violet/20% Ethanol (Sigma).
12. 2% BSA/PBS (Sigma).
13. Opti-MEM (GIBCO).
14. Lipofectamine (Invitrogen).
15. Clostridium perfringens neuraminidase (Boehringer).
16. 25 mM Citrate pH4.5 (Sigma).
17. 3,3′,5,5′ tetramethyl benzidine hydrochloride (Sigma).
18. 30% H₂O₂ (Sigma).
19. 0.1 N H₂SO₄ (Sigma).
20. Dithiothreitol (DTT).
21. Coomassie Brilliant Blue (Sigma).
22. Enzyme-linked immunosorbent assay (ELISA) reader (Fisher).
23. Dounce homogenizer.
24. PM10 membrane filter (Millipore).
25. Q15 Sartorius ion exchange column.
26. ELISA buffer: (49 mL) 25 mM Citrate, pH 4.5, (1 mL) 7 mg/mL 3,3′,5,5′ tetramethyl benzidine
hydrochloride (freshly made), and (50 g/mL) 30% H₂O₂.
27. Plaque overlay agar: solution A, 1.5% Noble Agar/DW; solution B, 2×DMEM, 2× Penicillin/Streptomycin, 2% fetal bovine serum (FBS). Solution A and B mixed 50:50.

Transfection Process Steps

Step #1 of the procedure is to grow stocks of the parental mutant virus vRB12.

The SARS-2 COVID-19 Spike 1 glycoproteins from selected mutant strains can be cloned into plasmid pRB21 and purified by commercial providers using an appropriate standard transfection technique for the size gene. An example is given below.

Step #2 involves plating CV-1 cells for generation of the viruses in supplemented DMEM (5% FBS+1× penicillin/streptomycin).
a) Plate CV-1 cells in 6-well plates 24 hours prior to inoculation in 2-3 mL of media volume
b) When cells are 60-80% confluent, wash cells 2× with serum-free DMEM and infect with 10⁴ pfu or vRB12 in 0.8 mL of Opti-MEM.
c) Incubate at 37° C. and 5% CO₂.

Step #3 involves transfection of vRB12 infected CV-1 cells with pRB21 plasmids
a) At 2-4 hours post-infection, prepared DNA so transfection can be done in this timeframe.
b) Mix 0.5-2.0 g plasmid DNA in 100 pL of Opti-MEM with 5 pL Lipofectamine and incubate at RT for 30 minutes.
c) Add liquid-plasmid compound to CV-1 cells using gentle agitation
d) Incubate at 37° C. for 4-16 hours, then add 1 mL DMEM supplemented with 10% FBS and Penicillin/Streptomycin
e) Incubate at 37° C. for 2-3 days
f) When CPE is observed, harvest supernatants and cells using a 1 mL pipette and gentle agitation of the monolayer

Step #4 involves plaque purification of rVV
a) Plate CV-1 cells in 6-well plates and incubate at 37° C. and 5% CO2 until 90% confluent
b) Perform serial 10-fold dilutions from transfected cell culture from undiluted to 10⁻⁵
c) Allow viruses to adsorb for 1 hour in incubator.
d) Aspirate inoculum and apple 2 mL agar overlay with supplemented DMEM+antibiotics
e) Allow overlay to solidify in hood for 10 minutes, then return to incubator for 2-3 days.
f) At days 2 or 3, large, clear plaques with inserts should be visible by comparison to control (negative vRB12) and resemble positive controls
g) Incubate for another 1-4 hours prior to selecting plaques
h) Confirm plaques my light microscopy and pick at least 5 of the larger, isolated plaques for each recombinant
i) Amplify plaques using CV-1 cells in 24-well plates using 1 mL volume. Use a small pipette to insert into the overlay.
j) Incubate for 2-3 days to grown small stocks and monitor for CPE.

Production Scale-Up and Purification of Final Glycoprotein

The desired protein expression can be confirmed by Western Blot technique. After confirmation, one mini-stock of this can be used for seed virus stock using standard seeding and culture techniques. After harvesting, save working stock in aliquots of 1 mL and store at −20° until ready to infect CV-1 cells.

Step #4 is the production of large amounts of COVID-19 gp using Vero E6 cells in Roller bottles.
a) Plate Vero E6 in supplemented DMEM in 6-well plates as described.
b) After 80% confluency, inoculate at m.o.i. of at least 3.
c) Harvest cells when detached by agitation
d) Decant cells in centrifuge bottle and spin at 4° C. at 250 g for 15 minutes
e) Decant supernatant, wash and resuspend infected CV-1 cells in 10 mM Tris HCl at pH 8.0
f) Homogenize on ice in Dounce homogenizer at least 100 Dounce cycles
g) Add sucrose to bring solution to 70% w/v in sucrose. Overlay tube with 10 mM Tris HCl at pH 8.0
h) Membranes are floated by flotation centrifugation and pelleted by high (82,700 g) for 60 minutes at 4° C.
i) Resuspend membrane fraction in 10 mM Tris HCl at pH 8.0, 10 mM CaCl₂, with 50 mg/mL MgCl₂ containing 50 mg/mL TPCK-trypsin and 1 U/mL DNAase to separate ectodomains from membranes
j) Incubate at standard temp and CO2 for 30 minutes
k) Pellet membrane by centrifugation at 150,000 g for 10 minutes
I) Harvest supernatant
m) Incubate at standard temp and CO2 for 30 minutes
n) Overlay mixture on 1-30% sucrose gradient in 10 mM Tris HCl at pH 8.0 and centrifuge at 180,000 g for 16 hours
o) Purify Spike gp further by binding to a 015 Sartorius ion exchange column in 10 mM Tris HCl at pH 8.0 and eluting in 150 nM NaCl in 10 mM Tris HCl at pH 8.0
p) Confirm structure and antigenicity by Western Blot and/or ELISA assays

Vaccine Adjuvant Review

Viral subunit proteins are antigenic, but unless combined with other compounds to stimulate the immune response (adjuvants), they are poorly immunogenic. In order to stimulate innate immunity, higher titers of both serum IgG and mucosal IgA, and induce clones of CD4+ and CD8+ CTL with receptors specific for viral proteins, an adjuvant must be employed.

The original adjuvants were aluminum salts. (Freund's adjuvants, complete or incomplete). These act to stabilize and preserve the antigens from premature degradation, and attract macrophages and DC to the injection site. Over the years, new adjuvants became available as advances in immunology and molecular biology gave rise to new forms of immune stimulants.

An ideal adjuvant should protect the antigens without interfering with their structure, attract antigen-presenting cells (APC), and stimulate immune response without undue toxicity. Bacterial toxins were developed as potent adjuvants, but toxic effects, including partial paralysis, led to their being discarded. Bacterial flagellin protein lacks the toxic effect of many bacterial cell wall extracts, and has been proven safe for vaccine injected via parenteral route, but its safety by the intranasal route is unproved. Chitosan, a mucopolysaccharide from crustaceans, can cause reactions in persons allergic to shellfish. Allergic reactions to substances delivered to the respiratory tract are especially hazardous.

Another category of adjuvants with many properties suitable for intranasal administration are Liposome bodies. These are self-assembling lipid vesicles with aqueous cores in sizes from 10-300 nm, similar to viruses. They can be linked to Toll-Like-Receptor (TLR) ligands for added adjuvant power. TLR ligands activate both innate and adaptive immune mechanisms, leading to cytokine release and effector cells up-regulating cytolytic activity (Perforin and Granzyme B genes).

SF-10 is a compound of synthetic human pulmonary surfactant or its bovine equivalent, Surfacten™, a phospho-lipoprotein made by type 2 alveolar cells, with a carboxy vinyl polymer as a viscosity improver. SF-10 effectively induces Flu A anti-HA neutralizing IgA antibodies in nasal and lung washes as well as IgG in sera. SF-10 effectively delivers antigen to DC and promotes cross-presentation to CTL, yielding high numbers of effector CD4+ and CD8+ cells specific for the viral antigen. SF-10+HA up-regulated perforin and Granzyme B in splenic cells after intranasal administration. This was proven experimentally after depletion of CTL deprived animals of protection after SF-10+Flu A HA i.n. vaccination. CD4+ cells were more critical than CD8+ cells in this protection, so the addition of SF-10 to a suspension of SARS-2/COVID-19 Spike antigens may impart critical advantages for a SARS-2/COVID-19 prime-boost vaccine strategy.

Advantages of Using SF-10 as Intranasal Adjuvant for SARS-2/COVID-19
a) Induction of polymeric IgA in the Upper Respiratory Tract Mucus-Associated Lymph Tissues (MALT)
b) Induction/boost of IgG neutralizing sera in blood
c) Induction of CD4+ and CD8+ CTL in spleen, increasing the # and quality of those induced by the prime injection of the VRP particles carrying RBD antigen [
d) Long-lasting immunity in both the MALT and Bronchus-Associated Lymph Tissues (BALT) e) SF-10 has been safety used as an adjuvant for human Flu A vaccines administered by the i.n. route.

SF-10 Preparation

SF-10 is made using commercially available Surfacten™ (Mitsubishi Pharma, Tokyo, Japan), plus 1,2 Dipalmitoyl1-phoshphtidylcholine (DPPC), and palmitate (PA). Synthetic human surfactant (SSF), is available for Nippon Fine Chemical (Osaka, Japan). Synthetic SP-related peptides (>80% grade), are available from Greiner (Frickenhausen, Germany). Carboxy vinyl polymer is available from Sigma-Aldrich (St. Louis, Mo.).

SSF is prepared by mixing the three lipids, DPPC, PG, and PA, plus various peptides (below) at a molar ratio of 75:25:30:0-6, respectively. SSF samples at 4 mg/mL can then be lyophilized for storage.

Amino Acid Sequence of Peptides Comprising SF-10

SP-B
(1-25)
FPIPLPYCWLCRALIKRIQAMIPKG
SP-B
(20-60)
AMIPKGALAVAYAQVCRVVPLVAGGICQCLAERYSVILLDT
SP-B
(64-80)
RMLPQLVCRLYLRCSMD
KL4
SP-C-type peptide
KLLLLKLLLLKLLLLKLLLLK
SP-C
(1-35)
FGIPCCPVHLKRLLIVVVVVVLIVVVIVGALLMGL
SP-C
(1-12)
FGIPCCPVHLKR
SP-C
(1-19)
FGIPCCPVHLKRLLIVVVV
SP-C
(13-35)
LLIVVVVVVLIVVVIVGALLMGL
SP-CL11
PVHLKRLLLLLLLLLLL
SP-CL16
PVHLKRLLLLLLLLLLLLLLLL
K6L16
KKKKKKLLLLLLLLLLLLLLLL

Mixing of SARS-2/COVID-19 Spike Glycoprotein and SF-10

SARS-2/COVID gp is treated for 3 minutes with a sonic oscillator followed by upside-down mixing every 30 minutes for 2 hours at RT, then stored at 4° C.

A mixture of SSF and an appropriate dose (75 ug), of SARS-2/COVID-Spike 1/2 gp is incubated at 42° C., the critical temperature of Surfacten lipids, for 10 minutes with gentle mixing, followed by freezing at −75° C., and then lyophilized. Lyophilized SARS-2/COVID-19 Spike ½ gp+SSF is dissolved in sterile saline and added to an atomizer unit and stored at 4° C. before use.

Administration of SARS-2/COVID-19 Spike Glycoprotein and SF-10

The atomizer is removed from cold storage, shaken, and the cap is removed. The subject places the atomizer at one nostril and depressed the plunger while inhaling through the nose.

Prime-Boost Strategy of Novel SARS-2/COVID-19 Vaccine

Prime-boost refers to the administration of multiple antigens, in sometimes varying formulations and routes of administration, in order to increase protection against viral challenge. For the invention described herein, the plan is:
1. Administration by intramuscular or intradermal hypodermic needle injection of the VRP carrying the SARS-2/COVID-19 Spike 1-RBD.
2. Approximately 10 days later, administration by intranasal route of the described SARS-2/COVID-19 mutant strain(s) Spike 1 glycoproteins+SF-10.
3. To provide protection from emergent mutant SARS-2/COVID-19 strains with new Spike 1 sequences, step #2 may be repeated using new plasmid constructs encoding the mutant gp Spike #sequence

The goal of the strategy is to stimulate a strong, multi-layered immune response in both the Upper and Lower Respiratory Tracts. Further Investigations with animal subjects may require some modification of the described dosing or administration plan.

Addressing Mutations and Emergent Strains of SARS-2/COVID-19

While DNA viruses are inherently stable, RNA viruses exhibit a high level of mutations. This is due to the relative accuracy of DNA polymerase (error rate of 1 in 10\hat{ }e7 base pairs) to RNA polymerase (error rate 1 in 10\vec{ }e4 base pairs). While many of these errors are fatal to the virion, some, especially those occurring in envelope gp antigens, can allow escape by inhibiting binding of neutralizing antibody, or CTL recognition of an epitope.

IFA exhibits considerable antigenic drift (moderate changes in genome) and also antigenic shift (dramatic changes in genome leading to impact on immune responses). Antigenic drift mutations may or may not impact immunity, but antigenic shift mutations lead to emergent strains, requiring new vaccine formulations for protection. The 2017 IFA vaccine had an estimated protective rate of 36%

SARS-2/COVID-19 exhibits moderate mutation rates compared with IFA, but this could be due to its relatively recent emergence, and lack of selective pressure from vaccine or natural immunity. There are several strains circulating, with unknown impact on disease severity or future vaccine development.

The traditional method of overcoming IF mutations is through new annual vaccine formulations to induce a new range of NAB against HA targets. However, this approach has several drawbacks:
a) The process of producing LAIV in chicken eggs may introduce new epitopes
b) Individuals who are allergic to eggs must have vaccines produced by other methods
c) The levels of serum NAB to HA may not confer sufficient protection against the emergent strain

Serum NAB may not be the ultimate goal of a vaccine protective against emergent strains. There is a consensus among thought leaders of respiratory virus vaccine development that serum NAB is only part of an overall protective strategy. Critical elements of this strategy include:
a) Polymeric IgA antibodies in the URT to trap and neutralize virus before it gains entry to the LRT
b) CD4⁺ T cells of the Follicular-Helper and Helper-Inducer subsets for induction of high titers of neutralizing IgG
c) CD8⁺ T cells recognizing conserved viral proteins expressed by infected cells, especially from the NP capsid. These can be broadly cross-protective in the case of IFA

A Multi-Part Prime-Boost Strategy as Described Herein could be Capable of Achieving these Goals:
1) The first injection of VRP with SARS-2/COVID-19 RBD could induce both neutralizing antibodies and CTL against conserved elements of the RBD.
2) The first nasal administration could increase the levels of polymeric IgA against a wider range of Spike gp targets besides the RBD.
3) Additional plasmids encoding the mutant emergent strain Spike sequence could be constructed and used as annual intranasal boosts.

What is claimed is:

1. A composition for preventing infection by the SARS-2/COVID-19 coronavirus in a subject in need thereof, comprising administering a recombinant DNA molecule coding for Alphavirus Virus-Like Replicative Particles containing a SARS-2/COVID-19 Spike 1 glycoprotein Receptor-Binding Domain transgene to the subject.

2. The recombinant DNA molecule Alphavirus-Like Replicative Particles of claim 1, wherein the alphavirus structural proteins are derived from Venezuelan Equine Encephalitis (VEE), Sindbis, or Semliki Forest Virus structural proteins.

3. The recombinant DNA molecule Virus-Like Replicative Particles of claim 1, wherein the Alphavirus is derived from the VEE³⁰⁰⁰ sequence modified by deletion of a sequence in Envelope gene 3 and a second site resuscitation insertion in Envelope gene 1, or other structural polyprotein-coding sequence comprises one or more attenuating mutations with the effect of decreased toxicity and enhanced immunogenicity.

4. The recombinant DNA molecule Virus-Like Replicative Particles of claim 1, wherein the transgene encoding the Receptor-Binding Domain of the SARS-2/COVID-19 is inserted into the Alphavirus sequence downstream of a 26S sub-genomic promoter.

5. The recombinant DNA molecule of claim 1, where an Internal Ribosome Entry Sequence (IRES), derived from an Enterovirus 71 sequence, allowing for amplified expression of the transgene, is inserted between the 26S sub-genomic promoter and the transgene.

6. The recombinant DNA molecule of claim 1, where the transgene comprises the receptor-binding domain sequence of a SARS-2/COVID-19 Spike 1 glycoprotein after the IRES sequence.

7. The recombinant DNA molecule of claim 1, where (i) a T7 bacteriophage promoter inserted to the 5′ terminus of the DNA molecule, (ii) a poly-Adenine chain is attached to the 3′ terminus of the DNA molecule, (iii) the DNA molecule is ligated with ligase.

8. A method of manufacturing semi-replication competent, semi-defective alphavirus replicon particles, comprising transfection into mammalian cells (i) the recombinant DNA molecule of claim 1 carrying a SARS-2/COVID-19 RBD transgene, under conditions whereby the alphavirus protein-coding sequence is transiently expressed from the DNA molecule to produce alphavirus structural proteins and infectious, (ii) and helper plasmids with the corresponding Alphavirus glycoprotein and nucleocapsid genes, (iii), and where all three plasmids are transfected into mammalian cells so replication-semi-defective alphavirus replicative particles are produced.

9. The method of claim 8, where the glycoprotein and capsid sequences of an Alphavirus are inserted into separate helper plasmids.

10. The method of claim 8, where the Alphavirus glycoprotein and capsid sequence plasmids 5′ and 3′ termini are fused together into circular plasmids.

11. The method of claim 8, wherein the recombinant DNA molecule is electroporated or transfected by other means into a population of mammalian cells.

12. The method of claim 8, where the Alphavirus Replicon Particles are harvested and purified before aliquoting in a sterile container.

13. The method of claim 8, wherein the Alphavirus Replicon Particles are injected into the dermis or muscle of a subject via a hypodermic syringe in a sufficient amount to elicit a protective immune response.

14. A composition for preventing infection by the SARS-2/COVID-19 coronavirus in a subject in need thereof, comprising administering SARS-2/COVID-19 Spike 1 glycoprotein subunits, wherein the SARS-2/COVID-19 Spike 1 glycoprotein subunits are produced by: (i) Copying DNA sequences of several different mutant strains of SARS-2/COVID-19 envelope Spike 1 glycoprotein genes into a plasmid, (ii) Inserting a T7 bacteriophage promoter to the 5′ terminus of the plasmid; (iii) Adding a poly-adenine chain to the 3′ terminus of the plasmid following the SARS-2/COVID-19 envelope glycoprotein sequence, (iv) using ligase to fuse the 5′ and 3′ termini of the linear plasmid to produce a circular plasmid.

15. The method of claim 14, wherein the circular plasmid coding for the SARS-2/COVID-19 envelope Spike 1 glycoprotein gene is transfected into a living mammalian cell culture.

16. The method of claim 14, wherein the transfection is accomplished by electroporation or lipofectamine.

17. The method of claim 11, wherein the resulting SARS-2 glycoproteins are harvested and purified by centrifugation, filtration, and sucrose density separation.

18. A composition for preventing infection by the SARS-2/COVID-19 coronavirus in a subject in need thereof, comprising administering SARS-2/COVID-19 Spike 1 glycoprotein subunits with an adjuvant SF-10, wherein the adjuvant SF-10 units are produced by organic chemical synthesis.

19. The method of claim 16, wherein the harvested and purified SARS-2/COVID-19 Spike 1 glycoprotein subunits are combined with SF-10 adjuvant to form a mixture.

20. The method of claim 16, wherein the SARS-2/COVID-19 Spike 1 glycoprotein subunits combined with SF-10 adjuvant are administered by intranasal inhalation in a sufficient amount to elicit a protective immune response.


SEQUENCE LISTING (4 sequences)

SEQ ID NO: 1 - DNA, 3826 bp, Artificial Sequence
  DNA encoding spike protein
aactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgctaaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtggtctccctcagggtttttcggctttagaaccattggtagatttgccaataggtattaacatcactaggtttcaaactttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaatattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttaaaggttttaattgttactttcctttacaatcatatggtttccaacccacttatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgtagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 2 - DNA, 3844 bp, Artificial Sequence
  DNA encoding spike protein
  misc_feature (267)..(267): n is a, c, g, or t
gatgttcttgttaacaactaaacgaacaatgtttgtttttcttgttttattgccactagtctctagtcagtgtgttaatcttagaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgntaaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtggagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggttttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatggtctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattaccggtatagattgtttaggaagtctaatctcaaaccttttgagagagatatttcaactgaaatctatcaggccggtagcaaaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcgtcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaaaatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 3 - DNA, 3822 bp, Artificial Sequence
  Complementary DNA sequence
atgtttgtttttcttgttttattgccactagtctctagtcagtgtgttaattttacaaacagaactcaattaccctctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaattatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagtgaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaacgattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttaaaggttttaattgttactttcctttacaatcatatggtttccaacccacttatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaatcaggttgctgttctttatcagggtgttaactgcacagaagtccctgttgctattcatgcagatcaacttactcctacttggcgtgtttattctacaggttctaatgtttttcaaacacgtgcaggctgtttaataggggctgaatatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctattaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcatttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 4 - DNA, 11447 bp, Artificial Sequence
  DNA Plasmid Sequence
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgttcccgttccagccaatgtatccgatgcagccaatgccctatcgcaacccgttcgcggccccgcgcaggccctggttccccagaaccgacccttttctggcgatgcaggtgcaggaattaacccgctcgatggctaacctgacgttcaagcaacgccgggacgcgccacctgaggggccatccgctaagaaaccgaagaaggaggcctcgcaaaaacagaaagggggaggccaagggaagaagaagaagaaccaagggaagaagaaggctaagacagggccgcctaatccgaaggcacagaatggaaacaagaagaagaccaacaagaaaccaggcaagagacagcgcatggtcatgaaattggaatctgacaagacgttcccaatcatgttggaagggaagataaacggctacgcttgtgtggtcggagggaagttattcaggccgatgcatgtggaaggcaagatcgacaacgacgttctggccgcgcttaagacgaagaaagcatccaaatacgatcttgagtatgcagatgtgccacagaacatgcgggccgatacattcaaatacacccatgagaaaccccaaggctattacagctggcatcatggagcagtccaatatgaaaatgggcgtttcacggtgccgaaaggagttggggccaagggagacagcggacgacccattctggataaccagggacgggtggtcgctattgtgctgggaggtgtgaatgaaggatctaggacagccctttcagtcgtcatgtggaacgagaagggagttaccgtgaagtatactccggagaactgcgagcaatggtcactagtgaccaccatgtgtctgctcgccaatgtgacgttcccatgtgctcaaccaccaatttgctacgacagaaaaccagcagagactttggccatgctcagcgttaacgttgacaacccgggctacgatgagctgctggaagcagctgttaagtgccccggaaggaaaaggagatccaccgaggagctgtttaaggagtataagctaacgcgcccttacatggccagatgcatcagatgtgcagttgggagctgccatagtccaatagcaatcgaggcagtaaagagcgacgggcacgacggttatgttagacttcagacttcctcgcagtatggcctggattcctccggcaacttaaagggcaggaccatgcggtatgacatgcacgggaccattaaagagataccactacatcaagtgtcactccatacatctcgcccgtgtcacattgtggatgggcacggttatttcctgcttgccaggtgcccggcaggggactccatcaccatggaatttaagaaagattccgtcacacactcctgctcggtgccgtatgaagtgaaatttaatcctgtaggcagagaactctatactcatcccccagaacacggagtagagcaagcgtgccaagtctacgcacatgatgcacagaacagaggagcttatgtcgagatgcacctcccgggctcagaagtggacagcagtttggtttccttgagcggcagttcagtcaccgtgacacctcctgttgggactagcgccctggtggaatgcgagtgtggcggcacaaagatctccgagaccatcaacaagacaaaacagttcagccagtgcacaaagaaggagcagtgcagagcatatcggctgcagaacgataagtgggtgtataattctgacaaactgcccaaagcagcgggagccaccttaaaaggaaaactgcatgtcccattcttgctggcagacggcaaatgcaccgtgcctctagcaccagaacctatgataacctttggtttcagatcagtgtcactgaaactgcaccctaagaatcccacatatctaaccacccgccaacttgctgatgagcctcactacacgcacgagctcatatctgaaccagctgttaggaattttaccgtcaccgaaaaagggtgggagtttgtatggggaaaccacccgccgaaaaggttttgggcacaggaaacagcacccggaaatccacatgggctaccgcacgaggtgataactcattattaccacagataccctatgtccaccatcctgggtttgtcaatttgtgccgccattgcaaccgtttccgttgcagcgtctacctggctgttttgcagatctagagttgcgtgcctaactccttaccggctaacacctaacgctaggataccattttgtctggctgtgctttgctgcgcccgcactgcccgggccgagaccacctgggagtccttggatcacctatggaacaataaccaacagatgttctggattcaattgctgatccctctggccgccttgatcgtagtgactcgcctgctcaggtgcgtgtgctgtgtcgtgccttttttagtcatggccggcgccgcaggcgccggcgcctacgagcacgcgaccacgatgccgagccaagcgggaatctcgtataacactatagtcaacagagcaggctacgcaccactccctatcagcataacaccaacaaagatcaagctgatacctacagtgaacttggagtacgtcacctgccactacaaaacaggaatggattcaccagccatcaaatgctgcggatctcaggaatgcactccaacttacaggcctgatgaacagtgcaaagtcttcacaggggtttacccgttcatgtggggtggtgcatattgcttttgcgacactgagaacacccaagtcagcaaggcctacgtaatgaaatctgacgactgccttgcggatcatgctgaagcatataaagcgcacacagcctcagtgcaggcgttcctcaacatcacagtgggagaacactctattgtgactaccgtgtatgtgaatggagaaactcctgtgaatttcaatggggtcaaattaactgcaggtccgctttccacagcttggacaccctttgatcgcaaaatcgtgcagtatgccggggagatctataattatgattttcctgagtatggggcaggacaaccaggagcatttggagatatacaatccagaacagtctcaagctcagatctgtatgccaataccaacctagtgctgcagagacccaaagcaggagcgatccacgtgccatacactcaggcaccttcgggttttgagcaatggaagaaagataaagctccatcattgaaatttaccgcccctttcggatgcgaaatatatacaaaccccattcgcgccgaaaactgtgctgtagggtcaattccattagcctttgacattcccgacgccttgttcaccagggtgtcagaaacaccgacactttcagcggccgaatgcactcttaacgagtgcgtgtattcttccgactttggtgggatcgccacggtcaagtactcggccagcaagtcaggcaagtgcgcagtccatgtgccatcagggactgctaccctaaaagaagcagcagtcgagctaaccgagcaagggtcggcgactatccatttctcgaccgcaaatatccacccggagttcaggctccaaatatgcacatcatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc